(19) United States
(12) Patent Application Publication
(10) Pub. No.: US 2022/0380398 A1
(43) Pub. Date: Dec. 1, 2022

(54) MANGIFERIN AND PREPARATION METHOD THEREOF

(71) Applicant: Xi'an Kintai Biotech,Inc, Xi'an City (CN)

(72) Inventors: Yuchi Zou, Xi'an City (CN); Jinfeng Chen, Xi'an City (CN)

(73) Assignee: Xi'an Kintai Biotech,Inc, Xi'an City (CN)

(21) Appl. No.: 17/826,696

(22) Filed: May 27, 2022

(30) Foreign Application Priority Data
May 28, 2021 (CN) 202110590748.1

(51) Int. Cl. C07H 1/08; C07H 17/04; C07D 407/04

(52) U.S. Cl. CPC C07H 1/08; C07H 17/04; C07D 407/04

(57) ABSTRACT

The present disclosure belongs to the technical field of plant extraction, and particularly relates to mangiferin and a preparation method thereof. The present disclosure includes activated carbon treatment and extraction of raw materials successively to obtain the mangiferin; the raw materials are mango leaves and/or almond leaves; the extraction is absolute methanol extraction, and the extraction is conducted at 60-65° C. Results of examples indicate that mangiferin obtained by the preparation method of mangiferin provided by the present disclosure has a purity of 98% to 99.5%, and mangiferin yield is as high as 66.7% of total mangiferin content in the mango leaves or almond leaves. The preparation method of mangiferin provided by the present disclosure is simple to operate, which obtains high-yield and highly pure mangiferin while reducing production costs of the mangiferin, and realizes the industrial production of the mangiferin.

### CROSS REFERENCE TO RELATED APPLICATION

This patent application claims the benefit and priority of Chinese Patent Application No. 202110590748.1, filed on May 28, 2021, the disclosure of which is incorporated by reference herein in its entirety as part of the present application.

### TECHNICAL FIELD

The present disclosure belongs to the technical field of plant extraction, and particularly relates to mangiferin and a preparation method thereof.

### BACKGROUND ART

Mangiferin (MGF, C₁₆H₁₂O₄) is present predominantly in the fruit, leaf, and bark of mango (Mangifera indica) of the family Anacardiaceae, the rhizome and aerial part of Anemarrhena asphodeloides of the family Liliaceae, and the flower and leaf of Belamcanda chinensis of the family Iridaceae. MGF has anti-tumor, antiviral, antioxidant, anti-diabetic activity and other physiological activity. It has an excellent developing prospect because its safety is accepted. However, due to weak solubility and transmembrane permeability and low bioavaolability of MGF, the exertion of pharmacological action of MGF is restricted.

Mango leaf is a leaf of M. indica of the family Anacardiaceae. With abundant resources in Guangxi, it is a principal raw material for producing Mangguo Zhike Pian (Mango Anticough Tablets). Research shows that MGF is a primary active pharmaceutical ingredient for relieving cough and expelling phlegm in the mango leaf. The MGF content in the mango leaf is intensively around 3%. At present, there have been a plurality of methods for separating MGF from mango leaves, including ultrasonic extraction, microwave-assisted extraction, decoction method, and ester extraction and impurity removal method, but their processes are generally complicated, their extraction efficiency is not well, and MGF yield is low. For example, Zhang et al. extracted MGF from mango leaves using ultrasound-assisted extraction (Optimization of ultrasound-assisted extraction of mangiferin from mango leaves with response surface methodology. Northwest Pharmaceutical Journal, 2014, 29(3)). Dried mango leaves were pulverized and sieved through 10- to 120-mesh screens; 2 g of the powder was weighed in a reactor of an ultrasonic extractor each time, sonicated with a certain amount of ethanol solution, and filtered off with suction; a filtrate was measured, 100 M filtrate was accurately pipetted into a 10 mL volumetric flask, and diluted with mobile phase methanol/2 mL·L⁻¹ phosphoric acid solution (20:80). Finally, the extraction rate of MGF was only 4.04%. How to increase the MGF yield and reduce extraction costs becomes a problem to be solved.

### SUMMARY

An objective of the present disclosure is to provide a preparation method of mangiferin. The preparation method provided by the present disclosure is simple to operate, which increases the yield and purity of mangiferin, reduces extraction costs, and realizes the industrial production of the mangiferin.

To achieve the above objective, the present disclosure provides the following technical solution:

The disclosure provides a preparation method of mangiferin, including the following steps:

treating raw materials with activated carbon and extracting to obtain mangiferin;

where the raw materials are mango leaves and/or almond leaves; the extraction is absolute methanol extraction, and the extraction is conducted at 60-65° C.

Preferably, the raw materials and the absolute methanol may have a mass-volume ratio of 1 kg:10 L to 1 kg:30 L; the raw materials and the activated carbon may have a mass ratio of 5:1 to 30:1.

Preferably, the absolute methanol extraction may be conducted in an extraction tank, and the absolute methanol extraction may be conducted under normal pressure.

Preferably, the extraction may be conducted 2-3 times, and each extraction may be conducted for 3-5 h.

Preferably, the extraction may be further followed by concentration; the concentration may be conducted in an external circulating concentrator.

Preferably, the concentration may include a first concentration and a second concentration;

the first concentration may be normal pressure concentration; the first concentration may be conducted at 65-80° C.;

the second concentration may be vacuum concentration; the second concentration may be conducted at 70-80° C., and concentration pressure may be in the range of −0.05 to −0.03 MPa.

Preferably, before the second concentration, the following steps may be further included: filtering a first concentrate obtained by the first concentration to obtain a coarse mangiferin crystal;

washing the coarse mangiferin crystal to obtain a yellow solid; and

dissolving the yellow solid, adding the activated carbon, and filtering.

Preferably, ethyl acetate and/or chloroform may be used in the washing.

Preferably, 50% ethanol may be used in the dissolving;

The yellow solid and the 50% ethanol may have a mass-volume ratio of 1 kg:50 L to 1 kg:100 L.

The present disclosure further provides mangiferin prepared by the foregoing preparation method, and the mangiferin has a purity of 98% to 99.5%.

The disclosure provides a preparation method of mangiferin, including the following steps: treating mango leaves or almond leaves as raw materials with activated carbon and extracting to obtain mangiferin; the extraction is absolute methanol extraction, and the extraction is conducted at 60-65° C. The preparation method provided by the present disclosure is simple to operate, by which chlorophyll and other impurities that hinder the dissolution of the mangiferin from the mango leaves or almond leaves are removed by the activated carbon; hot absolute methanol is used as an extraction solvent to dissolve the mangiferin in hot methanol, so that mangiferin yield is as high as 66.7% of total mangiferin content in the mango leaves or almond leaves. This reduces the production cost of the mangiferin, and the operation is simple.

Further, in the present disclosure, the extraction is followed by concentration, which oversaturates a concentration of mangiferin in a solution, increases the purity of the mangiferin, and obtains a pure product of mangiferin with a purity of at least 98%; meanwhile, specific production equipment including the extraction tank and the external circulating concentrator are used in the present disclosure to mass-produce the mangiferin and realizes the industrial production of the mangiferin.

### DETAILED DESCRIPTION OF THE EMBODIMENTS

The disclosure provides a preparation method of mangiferin, including the following steps:

treating raw materials with activated carbon and extracting to obtain mangiferin;

where the raw materials are mango leaves and/or almond leaves; the extraction is absolute methanol extraction, and the extraction is conducted at 60-65° C.

In the present disclosure, the raw materials may be mango leaves and/or almond leaves; the raw materials and the activated carbon may preferably have a mass ratio of 5:1 to 30:1, and further preferably 10:1. In the present disclosure, the raw materials may preferably be pulverized to 10- to 100-mesh, and further preferably 20-mesh, and mixed with the activated carbon. In the present disclosure, the activated carbon may preferably be powdered activated carbon. In the present disclosure, the activated carbon is irreplaceable and has decoloration and impurity removal effects. Chlorophyll may be removed well from the raw materials by treating the mango leaves or almond leaves with the activated carbon.

After the raw materials are mixed with the activated carbon, the absolute methanol extraction is conducted in the present disclosure to obtain a mangiferin-containing extract. In the present disclosure, the absolute methanol extraction may preferably be conducted in an extraction tank. The method provided by the present disclosure may use the extraction tank as an extractor, so as to favor the mass production of mangiferin and improve the industrial production of the mangiferin. In the present disclosure, the absolute methanol extraction may be conducted at 60-65° C., and preferably 65° C.; the absolute methanol extraction may preferably be conducted under normal pressure. In the present disclosure, the absolute methanol extraction may be conducted 2-3 times, and further preferably 2 times; the absolute methanol extraction may preferably be conducted for 3-5 h, and further preferably 4 h each time; a mass of the raw materials and a sum of volumes of absolute methanol used may preferably have a ratio of 1 kg:10 L to 1 kg:30 L, and further preferably 1 kg:14 L. In the present disclosure, the first absolute methanol extraction and activated carbon treatment are conducted at the same time, and operations are simple and convenient.

If the absolute methanol extraction is conducted 2 times, in a first extraction, the raw materials and the absolute methanol may preferably have a mass-volume ratio of 1 kg:5 L to 1 kg:10 L, and further preferably 1 kg:8 L; in a second extraction, the raw materials and the absolute methanol may preferably have a mass-volume ratio of 1 kg:4 L to 1 kg:8 L, and further preferably 1 kg:6 L; the absolute methanol in the first extraction and the second extraction may preferably have a volume ratio of 1:1 to 5:2, and further preferably 4:3.

If the absolute methanol extraction is conducted 3 times, in a first extraction, the raw materials and the absolute methanol may preferably have a mass-volume ratio of 1 kg:4 L to 1 kg:6 L, and further preferably 1 kg:5 L; in a second extraction, the raw materials and the absolute methanol may preferably have a mass-volume ratio of 1 kg:4 L to 1 kg:6 L, and further preferably 1 kg:5 L; in a third extraction, the raw materials and the absolute methanol may preferably have a mass-volume ratio of 1 kg:3 L to 1 kg:6 L, and further preferably 1 kg:4 L; the absolute methanol in the first extraction, the second extraction and the third extraction may preferably have a volume ratio of 8:6:4 to 6:5:3, and further preferably 5:5:4. In the present disclosure, mangiferin obtained by the absolute methanol extraction has fewer impurities, so as to favor subsequent treatment, increase the production efficiency of the mangiferin, and reduce production costs.

In case of multiple extraction, extracts obtained by each absolute methanol extraction may preferably be combined in the present disclosure.

In the present disclosure, the extract is subjected to a first concentration to obtain a first concentrate. In the absolute methanol extraction, a proportion of the first concentration may preferably be based on a total volume of the absolute methanol; the total volume of the absolute methanol and a volume of the first concentrate may preferably have a ratio of 10:1 to 20:1, and further preferably 14:1; the first concentration may preferably be normal pressure concentration; the concentration may preferably be conducted at 65-80° C., and further preferably 70° C.; in the present disclosure, the first concentration may preferably be conducted in an external circulating concentrator, favoring the mass production of the mangiferin and increasing the industrial production of the mangiferin. In the present disclosure, the first concentration oversaturates a concentration of mangiferin in a solution, favoring the precipitation of the mangiferin.

In the present disclosure, after the first concentration, the first concentrate is cooled to room temperature and filtered to obtain a coarse mangiferin crystal; the coarse mangiferin crystal is washed to obtain a yellow solid; the yellow solid is dissolved, mixed with the activated carbon, and filtered to obtain a mangiferin-containing filtrate. In the present disclosure, there is no special requirement for methods for filtering to obtain the coarse mangiferin crystal and the yellow solid, as long as well-known methods may be used. In the present disclosure, ethyl acetate and/or chloroform may preferably be used in the washing. If the ethyl acetate is used in the washing, the coarse mangiferin crystal and the ethyl acetate may preferably have a mass-volume ratio of 1 kg:5 L to 1 kg:20 L, and further preferably 1 kg:10 L; if the chloroform is used in the washing, the coarse mangiferin crystal and the chloroform may preferably have a mass-volume ratio of 1 kg:5 L to 1 kg:20 L, and further preferably 1 kg:10 L; in the present disclosure, 50% (v/v) ethanol may preferably be used in the dissolving; the yellow solid and the 50% (v/v) ethanol may preferably have a mass-volume ratio of 1 kg:20 L to 1 kg:100 L, and further preferably 1 kg:50 L; the yellow solid and the activated carbon may preferably have a mass ratio of 10:1 to 50:1, and further preferably 20:1. In the present disclosure, the activated carbon may preferably be powdered activated carbon. In the present disclosure, the mangiferin may be sufficiently dissolved in 50% ethanol; the activated carbon may conduct further decoloration to remove fat-soluble pigments, lightening a color of a yellow crystal and increasing the production efficiency of the mangiferin.

In the present disclosure, after dissolving and before adding the activated carbon, a resulting solution may preferably be held at 60-80° C., and further preferably 75° C.; holding time may preferably be 15-60 min, and further preferably 30 min. In the present disclosure, too long holding time is unfavorable for improving the production efficiency; too short time leads to insufficient dissolution of mangiferin crystals and has an impact on product yield. In the present disclosure, the holding condition may achieve sufficient dissolution of the mangiferin.

In the present disclosure, after adding the activated carbon and before filtering, a resulting solution may preferably be stirred for 5-30 min, and further preferably 15 min; the filtering may preferably be conducted while hot.

In the present disclosure, the mangiferin-containing filtrate obtained by filtering after adding the activated carbon is subjected to a second concentration to obtain a second concentrate. A concentration ratio of the second concentrate may preferably be based on a total volume of ethanol for dissolving the coarse mangiferin crystal, and the ethanol and the second concentrate may preferably have a volume ratio of 2:1 to 10:1, and further preferably 4:1; the second concentration may preferably be vacuum concentration; the concentration may preferably be conducted at 65-80° C., and further preferably 70° C.; concentration pressure may preferably be in the range of −0.05 to −0.03 MPa, and further preferably −0.04 MPa; in the present disclosure, the second concentration may preferably be conducted in an external circulating vacuum concentrator to mass-produce the mangiferin, increasing the industrial production of the mangiferin. In the present disclosure, the second concentration may reduce an amount of ethanol, lower an ethanol concentration, favor the crystallization of the mangiferin, and increase mangiferin yield.

In the present disclosure, after the second concentration, the second concentrate may preferably be cooled to room temperature and filtered off with suction to obtain a pale yellow crystal, namely the mangiferin provided by the present disclosure; the filtering off with suction may preferably be vacuum filtration.

To further illustrate the present disclosure, the preparation method of mangiferin provided by the present disclosure and the mangiferin prepared by the preparation method provided by the present disclosure will be described in detail below in conjunction with examples, but they should not be construed as limiting the protection scope of the present disclosure.

### Example 1

1) 100 kg of crushed mango leaves (the mangiferin content was around 3%) were pulverized to less than 20-mesh, fed into an extraction tank, mixed with 10 kg of powdered activated carbon, and extracted in absolute methanol twice under reflux. Each extraction lasted for 4 h, the extraction temperature was 65° C., and the extraction pressure is normal pressure. In the first extraction, 800 L of methanol was added; in the second extraction, 600 L of methanol was added.

2) Extracts were combined, the combined extracted was concentrated to 100 L at 70° C. under normal pressure, and the concentrate was discharged and cooled to room temperature; the concentrate was filtered to obtain a coarse mangiferin crystal, with a purity of 80% and a mass of 2.9 kg.

3) The coarse crystal was washed with 30 L of ethyl acetate to remove ester-soluble impurities, and filtered to obtain a yellow solid, with a purity of 90% and a mass of 2.46 kg.

4) The yellow crystal was heated and dissolved in 200 L of 50% ethanol, held at 75° C. for 30 min, stirred with 0.2 kg of activated carbon for 15 min, and filtered while hot; the filtrate was concentrated to 50 L at 70° C. and −0.04 MPa, cooled to room temperature, and filtered off with suction to obtain a pale yellow crystal, with a purity of 99.15% and a mass of 2.03 kg.

### Example 2

The procedure was as in Example 1, except that 100 kg of crushed almond leaves (the mangiferin content was around 3%) were taken in step 1) to finally obtain a pale yellow crystal, with a purity of 98.81% and a mass of 2.11 kg.

### Example 3

The procedure was as in Example 1, except that 100 kg of a mixture of crushed mango leaves and almond leaves in a mass ratio of 1:1 (the mangiferin content was around 3%) were taken in step 1) to finally obtain a pale yellow crystal, with a purity of 98.86% and a mass of 2.08 kg.

### Example 4

The procedure was as in Example 1, except that 20 kg of powdered activated carbon was added in step 1) to finally obtain a pale yellow crystal, with a purity of 98.86% and a mass of 1.97 kg.

### Example 5

The procedure was as in Example 1, except that 8 kg of powdered activated carbon was added in step 1) and the absolute methanol extraction was conducted 2 times (each extraction lasted for 4 h) to finally obtain a pale yellow crystal, with a purity of 98.65% and a mass of 2.15 kg.

### Example 6

The procedure was as in Example 1, except that 10 kg of powdered activated carbon was added in step 1) and the absolute methanol extraction was conducted at 60° C. to finally obtain a pale yellow crystal, with a purity of 99.15% and a mass of 1.93 kg.

### Example 7

The procedure was as in Example 1, except that 700 L of methanol was added in the first extraction and 700 L of methanol was added in the second extraction in step 1) to finally obtain a pale yellow crystal, with a purity of 99.13% and a mass of 2.04 kg.

### Example 8

The procedure was as in Example 1, except that the coarse crystal was washed with 50 L of ethyl acetate to remove ester-soluble impurities in step 3) to finally obtain a pale yellow crystal, with a purity of 99.35% and a mass of 1.95 kg.

### Example 9

The procedure was as in Example 1, except that the coarse crystal was washed with 20 L of ethyl acetate to remove ester-soluble impurities in step 3) to finally obtain a pale yellow crystal, with a purity of 98.68% and a mass of 2.18 kg.

### Example 10

The procedure was as in Example 1, except that the coarse crystal was washed with 30 L of chloroform to remove ester-soluble impurities in step 3) to finally obtain a pale yellow crystal, with a purity of 99.05% and a mass of 2.03 kg.

### Example 11

The procedure was as in Example 1, except that the yellow crystal was heated and dissolved in 300 L of 50% ethanol in step 4) to finally obtain a pale yellow crystal, with a purity of 99.45% and a mass of 1.89 kg.

### Example 12

The procedure was as in Example 1, except that in step 4), after holding at 78° C. for 20 min, 0.15 kg of activated carbon was added to finally obtain a pale yellow crystal, with a purity of 98.85% and a mass of 2.01 kg.

### Comparative Example 1

The procedure was as in Example 1, except that raw materials were treated with alumina in step 1) to finally obtain a pale yellow crystal, with a purity of 92.56% and a mass of 1.89 kg.

### Comparative Example 2

The procedure was as in Example 1, except that 1 kg of powdered activated carbon was added in step 1) to finally obtain a pale yellow crystal, with a purity of 95.37% and a mass of 2.18 kg.

From the foregoing examples, mangiferin obtained by the preparation method of mangiferin provided by the present disclosure has a purity of 98% to 99.5%, and mangiferin yield is as high as 66.7% of total mangiferin content in the mango leaves or almond leaves. The preparation method of mangiferin provided by the present disclosure is simple to operate, which obtains high-yield and highly pure mangiferin while reducing production costs of the mangiferin; moreover, specific production equipment, including the extraction tank and the external circulating concentrator, realize the industrial production of the mangiferin.

Although the present disclosure is described in detail in the foregoing examples, they are only a part of, not all of, the examples of the present disclosure. Other examples can be obtained by persons based on the examples without creative efforts, and all of these examples shall fall within the protection scope of the present disclosure.

What is claimed is:

1. A preparation method of mangiferin, comprising the following steps:
mixing raw materials with activated carbon and conducting absolute methanol extraction to obtain a mangiferin-containing extract; wherein
the raw materials are mango leaves and/or almond leaves; the extraction is conducted at 60-65° C.;
the raw materials and the activated carbon have a mass ratio of 5:1 to 30:1;
the extraction is further followed by concentration;
the concentration comprises a first concentration and a second concentration; the first concentration is normal pressure concentration; the second concentration is vacuum concentration;
before the second concentration, the following steps are further comprised: filtering a first concentrate obtained by the first concentration to obtain a coarse mangiferin crystal;
washing the coarse mangiferin crystal to obtain a yellow solid; and
dissolving the yellow solid, adding the activated carbon, and filtering;
wherein ethyl acetate is used in the washing; 50% ethanol is used in the dissolving; and
after the second concentration, cooling a concentrate obtained by the second concentration to room temperature, and filtering off with suction to obtain a pale yellow crystal.

2. The preparation method according to claim 1, wherein the raw materials and the absolute methanol have a mass-volume ratio of 1 kg:10 L to 1 kg:30 L.

3. The preparation method according to claim 1, wherein the absolute methanol extraction is conducted in an extraction tank, and the absolute methanol extraction is conducted under normal pressure.

4. The preparation method according to claim 1, wherein the extraction is conducted 2-3 times, and each extraction is conducted for 3-5 h.

5. The preparation method according to claim 3, wherein the extraction is conducted 2-3 times, and each extraction is conducted for 3-5 h.

6. The preparation method according to claim 1, wherein the concentration is conducted in an external circulating concentrator.

7. The preparation method according to claim 6, wherein the first concentration is conducted at 70-80° C.; and
the second concentration is conducted at −80° C., and concentration pressure is in the range of −0.05 MPa to −0.03 MPa.

8. The preparation method according to claim 1, wherein the yellow solid and the 50% ethanol have a mass-volume ratio of 1 kg:50 L to 1 kg:100 L.
